(19) United States
(12) Patent Application Publication
(10) Pub. No.: US 2003/0118653 A1
(43) Pub. Date: Jun. 26, 2003

(54) Quick dissolving oral mucosal drug delivery device with moisture barrier coating

(75) Inventors: Li-Lan H. Chen, Edison, NJ (US); Alfred Liang, Edison, NJ (US); Xu Zheng, Edison, NJ (US); Hsueh-Ling Wu, East Windsor, NJ (US)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Lavipharm Laboratories Inc., East Windsor, NJ

(21) Appl. No.: 10/166,564

(22) Filed: Jun. 10, 2002

Related U.S. Application Data
(60) Provisional application No. 60/303,324, filed on Jul. 6, 2001.

(51) Int. Cl. A61K 9/14

(52) U.S. Cl. 424/484

(57) ABSTRACT

Quick dissolving oral mucosal drug delivery devices having a mucosal surface-coat-forming inner layer disposed between two moisture barrier coating layers for administering an active agent or combination of active agents to a subject are provided. Methods for making such quick dissolving oral mucosal drug delivery devices and to methods for using such quick dissolving oral mucosal drug delivery devices offering the measured and controlled release of an active agent or combination of active agents to a subject are also provided.

This application claims priority from U.S. Provisional Application Serial No. 60/303,324, filed Jul. 6, 2001; the disclosure of which is incorporated herein by reference as if set forth herein in its entirety.

The present invention relates to quick dissolving oral mucosal drug delivery devices having a mucosal surface-coat-forming inner layer disposed between two moisture barrier coating layers for administering an active agent or combination of active agents to a subject. The present invention also relates to methods for making such quick dissolving oral mucosal drug delivery devices and methods for using such quick dissolving oral mucosal drug delivery devices to provide a measured, controlled release of an active agent or a combination of active agents to a subject.

Prescription and over-the-counter medications and other pharmaceutical products have traditionally been administered through oral ingestion, nasal sprays, injections and suppositories. For example, many pharmaceutical dosage forms are administrated orally in the form of solid shaped articles such as tablets, pills, caplets and capsules that retain their shape under moderate pressure. Generally these dosage forms are designed to be swallowed whole or chewed to deliver the medication with adequate amounts of liquid. Some patients, particularly pediatric and geriatric patients, have difficulty swallowing or chewing such solid dosage forms. Certain patients such as children or animals often resist taking medications, and may try to hide such dosage forms in order to spit it out later. In addition, many pediatric and geriatric patients are unwilling to take such solid dosage forms because they have difficulty swallowing them even when liquids are consumed therewith. Furthermore, the availability of liquids at the time of administering medications may be limited for certain patients and may be restricted for certain diseases and/or treatments.

Chewable tablets provide some advantages over conventional tablets. Such chewable tablets, however, are not suitable for children wearing braces and the taste of certain active agents may be unpleasant and difficult to mask in a chewable tablet. In addition, the use of chewable tablets may not eliminate the desire or need to administer water or some other liquid therewith.

Furthermore, the standard oral dosage forms, such as tablets, pills, caplets, and capsules, are designed for short residence time in the mouth. Absorption of the active agent from these dosage forms typically occurs in the gastrointestinal (GI) tract, after the active agent has separated from the dosage form and dissolved in the gastric fluids. For some active agents, it is desirable to achieve absorption through a mucosal tissue in order to accelerate onset of the therapeutic effect.

Many active agents are poorly absorbed, even after they are dispersed in the stomach, because of low solubility or slow dissolution rate in the gastric fluids. Tablets may be formulated so as to be quick dissolving. These tablets are commonly placed on the tongue and disintegrate rapidly in the oral cavity. These dosage forms, however, are not fixed to a mucosal tissue and may move around in the mouth. Consequently, these dosage forms do not overcome the risk associated with choking or gagging that occurs with subjects having limited control of their swallowing reflexes.

Mucoadhesive, water soluble films with instant wettability for intraoral administration of cosmetically or pharmaceutically active ingredients have been suggested, which films rapidly dissolve/disintegrate upon application in the oral cavity. These mucoadhesive films exhibiting instant wettability tend to be hygroscopic resulting in storage and stability problems which may limit their shelf life. Also, some patients may experience difficulty self administering these mucoadhesive films because they are extremely sensitive to moisture. In particular, patients suffering from dyshidrosis, stress or other afflictions which result in chronic sweaty hands may experience difficulty handling these films given their inherent tendency to adhere to moist surfaces.

### Glossary

The following definitions are provided to facilitate an understanding of certain terms used frequently herein.

The term “coating solution” as used herein means a viscous and homogeneous mixture of hydrocolloids, active agents and other additives in a solvent. The coating solution is treated according to the method of the invention to form a film layer.

The term “disintegration time” as used herein means the time (in seconds) in which a film breaks when brought into contact with water or saliva.

The term “dissolving time” as used herein means the time (in seconds) in which not less than 80% of the film being tested is dissolved in an aqueous media or saliva.

The term “dry tack” as used herein is a quantitative value for the tackiness (in grams) of a dry film layer by Texture Analyzers (Model TAXT2i with 6 mm diameter stainless steel cylinder probe) from Texture Technologies Corp.

The term “hydration rate” as used herein means the speed of water absorption at 25° C. and 75% relative humidity in 24 hours.

The term “% elongation” as used herein is measured when the film snaps as sufficient force is applied so as to exceed the elastic limit.

The term “measured, controlled release” as used herein means that a predetermined dosage of an active agent or combination of active agents is administered to a subject.

The term “modulus” as used herein is a measurement of the stiffness of a film layer.

The term “mucosal surface-coat-forming” as used herein, as applied to a film layer, means that the subject film will rapidly lose its film structure and coat an oral mucosal surface (see FIG. 1) on contact, and may not thereafter be manually recovered or moved from the contact site.

The term “oral mucosal surface” as used herein includes lingual, sub-lingual, buccal, gingival and palatal surfaces; most preferably lingual, sub-lingual and buccal surfaces.

The term “permeation enhancer” as used herein means a natural or synthetic molecule which facilitates the absorption of a given active agent or combination of active agents through a mucosal tissue.

The term “quick dissolving” as used herein means a device which dissolves or disintegrates in the oral cavity of a subject within 1 to 600 seconds, more preferably within 1 to 60 seconds, most preferably in less than 30 seconds.

The term “release period” as used herein means the period of time subsequent to administration of a quick dissolving oral mucosal drug delivery of the present invention during which the delivery device releases an active agent or combination of active agents to a subject.

The term “subject” as used herein means an animal, preferably a mammal, most preferably a human.

The term “tensile strength” as used herein is the property of a film layer that requires a load to cause load deformation failure of the layer given in (psi).

The term “tear resistance” as used herein is the average force (in N) necessary to propagate a tear across a film layer or sheet under a specified rate of extension as defined in ASTM D1938 and is interpreted from the load time chart.

The term “thickness” as used herein by measurements in mil (a mil=one thousandth of an inch) is determined when a delivery device of the present invention is placed between two microscopic slides.

The term “water content” as used herein means the % residual water content per unit dosage of mucosal surface-coat-forming inner layer material of the present invention as measured according to the Karl Fisher method and expressed as percent of the dry weight of the film.

The term “wet tack” as used herein is a quantitative value for the tackiness (in grams) of a film layer after the addition of 10 ml of water to the surface thereof by Texture Analyzers (Model TAXT2i with 6 mm diameter stainless steel cylinder probe) from Texture Technologies Corp. The purpose of the wet tack analysis is to simulate the adhesion of the film layer upon contact with a moist mucosal surface.

### SUMMARY OF THE INVENTION

The present invention provides quick dissolving oral mucosal drug delivery devices which contain: (a) a mucosal surface-coat-forming inner layer disposed between (b) two moisture barrier coating layers; wherein the mucosal surface-coat-forming inner layer contains a water-soluble hydrocolloid or a combination of water-soluble hydrocolloids and an active agent or a combination of active agents and wherein the two moisture barrier coating layers contain a non-crosslinked polymer or a combination of non-crosslinked polymers and a moisture barrier modifier or a combination of moisture barrier modifiers. The active agents contained in the quick dissolving oral mucosal drug delivery devices of the present invention include: therapeutic agents, dietary supplements and hygiene aids.

The mucosal surface-coat-forming inner layers of the quick dissolving oral mucosal drug delivery devices of the present invention may further preferably contain one or more of a plasticizer or a combination of plasticizers, and a detackifier or a combination of detackifiers.

The mucosal surface-coat-forming inner layers of the quick dissolving oral mucosal drug delivery devices of the present invention may further optionally contain one or more of a taste modifying agent or a combination of taste modifying agents, an emulsifying agent or a combination of emulsifying agents, a buffering agent or a combination of buffering agents, a coloring agent or a combination of coloring agents and a preservative or a combination of preservatives.

The mucosal surface-coat-forming inner layers of the quick dissolving oral mucosal drug delivery devices of the present invention preferably have a thickness in the range of 1 to 50 mils.

The moisture barrier coating layers of the quick dissolving oral mucosal drug delivery devices of the present invention may further contain an anti-oxidant or a combination of anti-oxidants and/or a flavoring agent or a combination of flavoring agents.

The moisture barrier coating layers of the quick dissolving oral mucosal drug delivery devices of the present invention preferably have a thickness in the range of 1 to 25 μm.

The quick dissolving oral mucosal drug delivery devices provided by the present invention preferably dissolve or disintegrate in the oral cavity within 1 to 600 seconds, more preferably within 1 to 60 seconds, most preferably in less than 30 seconds.

The present invention also provides quick dissolving oral mucosal drug delivery devices which preferably contain: (a) a mucosal surface-coat-forming inner layer disposed between (b) two moisture barrier coating layers; wherein the mucosal surface-coat-forming inner layer contains a least one water soluble hydrocolloid, at least one active agent, at least one plasticizer and at least one detackifier; and wherein the two moisture barrier coating layers comprise at least one water soluble or dispersible film former and at least one moisture barrier modifier. The mucosal surface-coat-forming inner layer may further contain at least one member selected from the group consisting of taste modifying agents, emulsifying agents, buffering agents, coloring agents and preservatives. The moisture barrier coating layers may further contain at least one member selected from the group consisting of flavoring agents and anti-oxidants.

The present invention also provides methods for making quick dissolving oral mucosal drug delivery devices, including: (a) dissolving a hydrocolloid in a solvent to form a substantially homogeneous preparation; (b) adding to the preparation of (a), an active agent and at least one reagent selected from the group consisting of an emulsifer, a plasticizer, a taste modifier, a water soluble inert filler, a coloring agent, a preservative, a permeation enhancer, a stabilizer and a buffering agent to form a coatable or extrudable mixture; (c) forming a mucosal surface-coat-forming inner layer from the mixture of (b); and (d) applying a moisture barrier coating layer to the mucosal surface-coat-forming inner layer of (c).

The present invention also provides methods for administering an active agent or a combination of active agents to a subject including: (a) applying a quick dissolving oral mucosal drug delivery device of the present invention to an oral mucosal surface of the subject, wherein the quick dissolving oral mucosal drug delivery device contains a mucosal surface-coat-forming inner layer disposed between two moisture barrier coating layers; wherein the mucosal surface-coat-forming inner layer contains a water-soluble hydrocolloid, at least one active agent, at least one plasticizer and at least one detackifier; and wherein the two moisture barrier coating layers contains at least one water soluble or dispersible film former and at least one moisture barrier modifier.

The present invention also provides quick dissolving oral mucosal drug delivery devices which contain: (a) a mucosal surface-coat-forming inner layer encapsulated within (b) a moisture barrier coating layer; wherein the mucosal surface-coat-forming inner layer contains a water-soluble hydrocolloid or a combination of water-soluble hydrocolloids and an active agent or a combination of active agents and wherein the moisture barrier coating layer contain a non-crosslinked polymer or a combination of non-crosslinked polymers and a moisture barrier modifier or a combination of moisture barrier modifiers. The active agents contained in the quick dissolving oral mucosal drug delivery devices of the present invention include: therapeutic agents, dietary supplements and hygiene aids.

The present invention also provides quick dissolving oral mucosal drug delivery devices which contain: (a) a mucosal surface-coat-forming inner layer encapsulated by (b) a moisture barrier coating layer; wherein the mucosal surface-coat-forming inner layer contains a least one water soluble hydrocolloid, at least one active agent, at least one plasticizer and at least one detackifier; and wherein the moisture barrier coating layer comprises at least one water soluble or dispersible film former and at least one moisture barrier modifier. The mucosal surface-coat-forming inner layer may further contain at least one member selected from the group consisting of taste modifying agents, emulsifying agents, buffering agents, coloring agents and preservatives. The moisture barrier coating layer may further contain at least one member selected from the group consisting of flavoring agents and anti-oxidants.

### BRIEF DESCRIPTION OF THE DRAWING

There are shown in the drawings certain exemplary embodiments of the present invention as presently preferred. It should be understood that the present invention is not limited to the embodiments disclosed as examples, and is capable of variation within the spirit and scope of the appended claims.

In the drawings,

FIG. 1 is a depiction of the possible application sites in the oral cavity for the quick dissolving oral mucosal drug delivery devices of the present invention, namely the upper lip 1, the gingiva 2, the hard palate 3, the cheek 4, the lingual 5, the sublingual 6, and the lower lip 7;

FIG. 2 is a depiction of a cross-section of a preferred quick dissolving oral mucosal drug delivery device of the present invention;

FIG. 3 is a graph illustrating how the disintegration rate and dissolution rate can vary as a function of film layer thickness;

FIG. 4 is a process diagram for a preferred manufacturing method of the quick dissolving oral mucosal drug delivery devices of the present invention;

FIG. 5 is a process diagram for another preferred manufacturing method of the quick dissolving oral mucosal drug delivery devices of the present invention;

FIG. 6 is a process diagram for another preferred manufacturing method of the quick dissolving oral mucosal drug delivery devices of the present invention;

FIG. 7 is a process diagram for another preferred manufacturing method of the quick dissolving oral mucosal drug delivery devices of the present invention;

FIG. 8 is a graph illustrating the release profiles for the active agents incorporated into the mucosal surface-coat-forming inner layers of Examples 5-8; and,

FIG. 9 is a graph illustrating the moisture uptake of quick dissolving oral mucosal drug delivery devices of the present invention with various moisture barrier coating layers in comparison with a mucosal surface-coat-forming inner layer without a moisture barrier coating layer.

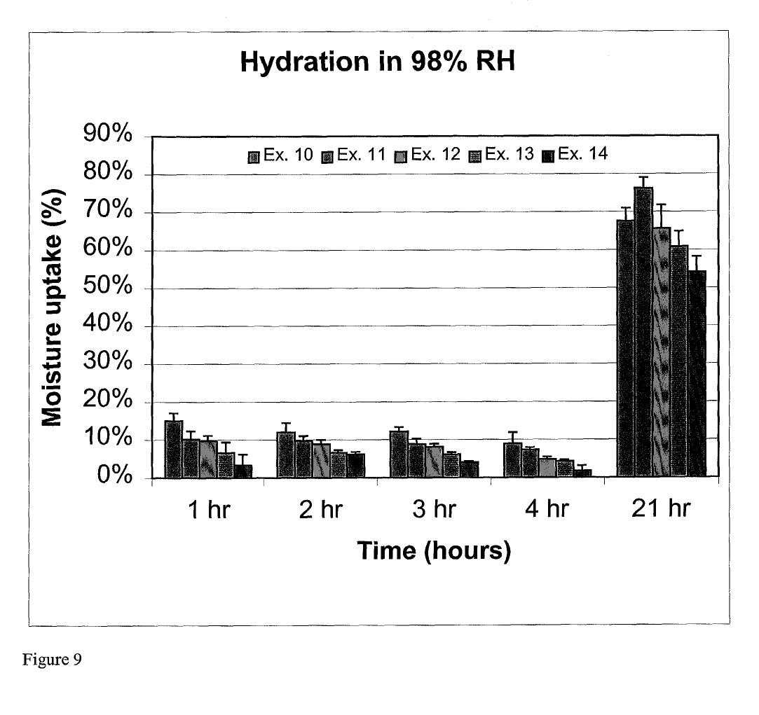

### DETAILED DESCRIPTION

The quick dissolving oral mucosal drug delivery devices of the present invention provide a drug delivery system which facilitates the release of the active agents without mastication or the need intake a fluid therewith (e.g. water).

In an embodiment of the present invention, quick dissolving oral mucosal drug delivery devices are provided in the form of a flexible, non-tacky, dry and conveniently packaged film. Once removed from the package and placed on an oral mucosal surface, all or a portion of the moisture barrier coating layers are melted and cleared away, exposing the mucosal surface-coat-forming inner layer to the oral mucosae. All or a portion of the moisture barrier coating layers may be melted and cleared away, for example, through oral cavity temperature and deformation thereof by the application of slight pressure on the delivery device against a mucosal surface, for instance pressing the delivery device to the gingiva or hard palate with the lingual surface of the tongue. Alternatively, all or a portion of the moisture barrier coating layers may be designed to disintegrate rapidly upon introduction into the oral cavity and exposure to the mucosal fluids present therein. Upon exposure to an oral mucosal surface the mucosal surface-coat-forming inner layer will hydrate substantially immediately to form a coating on the moist surface of the mucous membrane and then disintegrate and, or, dissolve to release the active agent or combination of active agents contained therein.

Preferred quick dissolving oral mucosal drug delivery devices of the present invention exhibit the following characteristics, namely (a) they should be sufficiently flexible to adapt to the surface of the oral mucosal tissue to which they are adapted to be administered, (b) they should be comfortable and unobtrusive during use, (c) they should be easy to administer to the site of application, (d) they should hydrate rapidly on the mucosal tissue once administered thereto, (e) they should be capable of providing a measured, controlled release of an active agent or a combination of active agents; (f) they should not cause irritation and (g) they should be completely dissolved and/or eroded at the end of the release period without the need for the physical removal of any residue.

The quick dissolving oral mucosal drug delivery devices of the present invention are intended to be inserted by the subject to be treated and do not require fitting by a physician. They can be easily inserted digitally by the subject or with the aid of an applicator.

The quick dissolving oral mucosal drug delivery devices of the present invention may be used as a vehicle for delivering a wide range of active agents to a subject. For example, the active agent may include small molecules (i.e., less than 1,000 daltons), proteins, nucleic acids including antisense molecules or other biological or synthetic molecules. Active agents suitable for use with the present invention include, but are by no means limited to, therapeutic agents, nutritional supplements and hygiene aids.

In an embodiment of the present invention, quick dissolving oral mucosal drug delivery devices are provided having a mucosal surface-coat-forming inner layer 100 disposed between two moisture barrier coating layers 110, see FIG. 2.

The mucosal surface-coat-forming inner layer of the quick dissolving oral mucosal drug delivery devices of the present invention preferably contain a hydrocolloid or a combination of hydrocolloids, an active agent or a combination of active agents, a plasticizer or a combination of plasticizers and a detackifier or a combination of detackifiers. The mucosal surface-coat-forming inner layer may also optionally contain taste modifying agents or a combination of taste modifying agents, a buffering agent or a combination of buffering agents, a coloring agent or a combination of coloring agents and a preservative or a combination of preservatives. Preferably, the mucosal surface-coat-forming inner layer has a thickness between 1 to 50 mil.

Hydrocolloids suitable for use in the mucosal surface-coat-forming inner layer of the quick dissolving oral mucosal drug delivery devices of the present invention include: water soluble non-gelling (at room temperature) natural polysaccharide or derivatives, water soluble non-gelling polypeptide or protein and synthetic hydrocolloids.

Examples of water soluble non-gelling (at room temperature) natural polysaccharide or derivatives suitable for use in the mucosal surface-coat-forming inner layer of the quick dissolving oral mucosal drug delivery devices of the present invention include: pectin and derivatives, guar gum, tragacanth gum, xanthan gum, gellan sodium salt, propyleneglycol alginate, starches (e.g., amylose, amylopectin), modified starches, hydroxyethyl starch, pullulan, carboxymethyl starch, gum ghatti, okra gum, karaya gum, dextrans, dextrins and maltodextrins, konjiac, acemannan from aloe, locust bean gum, tara gum, quince seed gum, fenugreek seed gum, scleroglucan, gum arabic, psyllium seed gum, tamarind gum, oat gum, carrageenans, succinoglucan, larch arabinogalactan, flaxseed gum, chondroitin sulfates, hyaluronic acid, curdlan, chitosan, deacetylated konjac, and rhizobium gum.

Examples of water soluble non-gelling polypeptide or protein suitable for use in the mucosal surface-coat-forming inner layer of the quick dissolving oral mucosal drug delivery devices of the present invention include: gelatins, albumins, milk proteins, soy protein, and whey proteins.

Examples of synthetic hydrocolloids suitable for use in the mucosal surface-coat-forming inner layer of the quick dissolving oral mucosal drug delivery devices of the present invention include: polyethylene-imine, hydroxyethyl cellulose, sodium carboxymethyl cellulose, carboxymethyl cellulose, hydroxypropyl cellulose, hydroxypropyl methyl cellulose, methyl cellulose, ethyl cellulose, polyacrylic acids, low molecular weight polyacrylamides and their sodium salts (carbomers), polyvinylpyrollidone, polyethylene glycols, polyethylene oxides, polyvinyl alcohols, pluronics, tetronics, other block co-polymers, carboxyvinyl polymers, and colloidal silicon dioxide.

The most preferred hydrocolloids suitable for use in the mucosal surface-coat-forming inner layer of the quick dissolving oral mucosal drug delivery devices of the present invention include hydroxypropyl methyl cellulose having a methoxy content of 19-30% and a hydroxpropyl content of 7 to 12% with a molecular weight of 50,000 to 250,000 daltons (Table 9).

Active agents suitable for use in the mucosal surface-coat-forming inner layer of the quick dissolving oral mucosal drug delivery devices of the present invention for human and or veterinary applications include: therapeutic agents, nutritional supplements and hygiene aids. Preferred active agents for use in the mucosal surface-coat-forming inner layers of the quick dissolving oral mucosal drug delivery devices of the present invention include nicotine, hydromorphone, oxybutynine, estradiol, famotidine, granisetron, hydrocortisone, loratadine, vinpocetine, buprenorphine, domperidone and loperamide.

Examples of therapeutic agents suitable for use in the mucosal surface-coat-forming inner layer of the quick dissolving oral mucosal drug delivery devices of the present invention include: analgesics, -adrenergic receptor blockers, anti-Alzheimer's disease medication, antianginal, antianxiety, antiarrythmics, antiarthritics, antibiotics, anticoagulants/thrombolytics, anticonvulsants/anti-Parkinson medication, anti-depressants, anti-diabetics, anti-diarrheal, anti-epileptics, anti-fungal, anti-gout, anti-heartworm medication for dogs, anti-histamines, anti-hypertensives, anti-inflammatories, anti-infectives, anti-migraines, anti-nasuants/anti-emetics, anti-neoplastics/anti-tumor active agents, anti-pruitics, anti-psychotics, anti-pyretics, anti-spasmodics, anti-virals, bronchial dilators/anti-asthmatics, calcium antagonists, cardiac agents, cardiotonics, central nervous system actives, contraceptives, coronary vasodilators, cough/cold remedies, dietary supplements (e.g., vitamins and minerals), diuretics, fertility active agents, flea control agents for animals (Ivermectin), H₂ receptor antagonists, herbal actives, hormones, hypoglycemics, hypolipidemics, muscle relaxants, ovulation stimulators, peptide active agents, polypeptide active agents, proteins (e.g., insulin, calcitonin, LHRH and the like), sedatives and hypnotics, sexual dysfunction active agents, sleep aids, smoking cessation aids, steroids and steroidals, tranquilizers, laxatives, ophthalmic preparations, nutritional supplements, breath fresheners, breath deodorants, saliva substitutes, antigingivitis agents, anti-cavity agents, anti-plaque agents, diagnostic indicators, local anesthetics, treatment agents for osteoporosis, treatment agents for hormone replacement, treatment agents for periodontal disease, antiseptics, corticosteroids, non steroidal anti-inflammatory agents, antiviral agents and vaccines.

Plasticizers suitable for use in the mucosal surface-coat-forming inner layer of the quick dissolving oral mucosal drug delivery devices of the present invention include: low molecular weight polyols (e.g., glycerin, propylene glycol); polyethylene glycols with molecular weight less than 1,000 daltons; polypropylene glycols with molecular weight of 200 daltons or less; glycol esters (e.g., propylene glycol monethyl ether); esters (e.g., sorbitol lactate, ethyl glycol); amines (e.g. triethanolamine); and sugars (e.g. sorbitol, sucrose).

Detackifiers suitable for use in the mucosal surface-coat-forming inner layer of the quick dissolving oral mucosal drug delivery devices of the present invention include: water insoluble polymers (e.g., cellulose acetate phthalate, polymethacrylate); lipids and fatty acids (e.g., carnauba wax, cetyl alcohol); inorganic diluents (e.g., calcium carbonate, talc); disintegrants (e.g., crosarmellose sodium, starch, microcrystalline cellulose); and, sugars (e.g., mannitol, xylitol, maltitol, lactose).

Taste modifying agents suitable for use in the mucosal surface-coat-forming inner layer of the quick dissolving oral mucosal drug delivery devices of the present invention include: flavoring agents, sweetening agents and taste masking agents. Examples of taste modifying agents suitable for use with the present invention include: the essential oils or water soluble extracts of menthol, wintergreen, peppermint, sweet mint, spearmint, vanillin, cherry, chocolate, cinnamon, clove, lemon, orange, raspberry, rose, spice, violet, herbal, fruit, strawberry, grape, pineapple, peach, kiwi, papaya, mango, coconut, apple, coffee, plum, watermelon, nuts, durian and green tea. Encapsulation of the active agent or combination of active agents may also be utilized to achieve taste masking of active agents that are bitter.

Buffering agents suitable for use in the mucosal surface-coat-forming inner layer of the quick dissolving oral mucosal drug delivery devices of the present invention include: acidulants and alkalizing agents. Examples of buffering agents suitable for use with the present invention include: citric acid, fumaric acid, lactic acid, tartaric acid, malic acid, as well as sodium citrate, sodium bicarbonate and carbonate, and sodium or potassium phosphate.

Coloring agents suitable for use in the mucosal surface-coat-forming inner layer of the quick dissolving oral mucosal drug delivery devices of the present invention include: FD & C coloring agents, natural coloring agents, and natural juice concentrates, pigments such as titanium oxide, silicon dioxide and zinc oxide.

Preservatives suitable for use in the mucosal surface-coat-forming inner layer of the quick dissolving oral mucosal drug delivery devices of the present invention include: anti-microbial agents and non-organic compounds. Examples of preservatives suitable for use with the present invention include sodium benzoate, parabens and derivatives, sorbic acid and its salts, propionic acids and its salts, sulfur dioxide and sulfites, acetic acid and acetates, nitrites and nitrates.

In a preferred embodiment of the present invention, the mucosal surface-coat-forming inner layer contains a hydrocolloid concentration in the range of 2 to 90% (of the dry weight of the layer), more preferably a concentration greater than 5% (of the dry weight of the layer).

In another preferred embodiment of the present invention, the mucosal surface-coat-forming inner layer contains (on a dry weight basis): 0.01 to 75% active(s); 1 to 40% plasticizer(s); 1 to 30% detackifier(s); 0 to 30% flavoring agent(s); 0 to 25% sweetener(s); 0 to 10% emulsifying agent(s); 0 to 10% buffering agent(s); 0 to 5% coloring agent(s); and, 0 to 5% preservative(s).

The moisture barrier coating layers of the quick dissolving oral mucosal drug delivery devices of the present invention may preferably contain a water soluble or dispersible film former or a combination of water soluble or dispersible film formers, a moisture barrier modifier or a combination of moisture barrier modifiers and a flavoring agent or a combination of flavoring agents. The moisture barrier coating layers may also optionally contain an anti-oxidant or a combination of anti-oxidants and/or a flavoring agent or a combination of flavoring agents.

Water soluble or dispersible film formers suitable for use in the moisture barrier coating layers of the quick dissolving oral mucosal drug delivery devices of the present invention include: thermoplastic polymers (e.g., hydroxypropylene cellulose, polyethylene oxide, polyethylene glycol); non-ionic synthetic polymers with moderate or poor mucoadhesive force (e.g., hydroxyethylcellulose, polyvinyl pyrrolidone, methyl cellulose, hydroxypropyl methyl cellulose, soluble starch, polyvinyl alcohol); and protein based film formers (e.g., zein, gluten, casein, whey protein, albumin, soy protein).

Moisture barrier modifiers suitable for use in the moisture barrier coating layers of the quick dissolving oral mucosal drug delivery devices of the present invention include: waxes, solid lipids and resins. Examples of moisture barrier modifiers suitable for use with the present invention include: hydrogenated castor oil, cetostearyl alcohol, cetyl alcohol, cetyl esters wax, glyceryl monosterate, glyceryl palmitostearate, lecithin, poloxamer, polyoxyethylene alkyl ethers, polyoxethylene stearates, sorbitan esters, stearyl alcohol, hydrogenated vegetable oil type I, carnauba wax, microcrystalline wax, nonionic emulsifying wax, white wax, yellow wax and shellac. The melting point of the moisture barrier modifiers used in the moisture barrier coating layers of the quick dissolving oral mucosal drug delivery devices of the present invention preferably have a melting point in excess of 45° C. The moisture barrier modifiers serve as a moisture barrier during storage. The moisture barrier modifiers may also serve to facilitate clearance of the dosage form from the site of application upon hydration thereof.

Flavoring agents suitable for used in the mucosal surface-coat-forming inner layer are also suitable for use in the moisture barrier coating layers of the quick dissolving oral mucosal drug delivery devices of the present invention and include: the essential oils or water soluble extracts of menthol, wintergreen, peppermint, sweet mint, spearmint, vanillin, cherry, chocolate, cinnamon, clove, lemon, orange, raspberry, rose, spice, violet, herbal, fruit, strawberry, grape, pineapple, peach, kiwi, papaya, mango, coconut, apple, coffee, plum, watermelon, nuts, durean and green tea.

Anti-oxidants suitable for use in the moisture barrier coating layers of the quick dissolving oral mucosal drug delivery devices of the present invention include: alpha tocopherol, butylated hydroxyanisole, butylated hydroxytoluene, propyl gallate and sodium metabisufite.

In a preferred embodiment of the present invention, the moisture barrier coating layers contain (on a dry weight basis): 10 to 90% water soluble or dispersible film former(s); 10 to 90% moisture barrier modifier(s); and, 1 to 40% flavoring agent(s).

The quick dissolving oral mucosal drug delivery devices of the present invention may release an active agent or a combination of active agents over a release period which is determined by a number of different factors. These factors include the dissolution/disintegration rate of the delivery device in the oral cavity and the thickness of the mucosal surface-coat-forming inner layer.

A quick dissolving oral mucosal drug delivery device having higher dissolution/disintegration rate should exhibit a shorter release period than an otherwise similar device with a lower dissolution/disintegration rate.

The thickness of the mucosal surface-coat-forming inner layer of the quick dissolving oral mucosal drug delivery devices of the present invention may be a factor in determining the rate of dissolution. A thick inner layer will dissolve more slowly than an otherwise similar thin inner layer. A thick inner layer may be desirable over a similar thin inner layer to facilitate larger dosages of an active agent or combination of active agents based on the relative holding capacity of such devices. FIG. 3 graphically represents the rate of disintegration and dissolution as a function of inner layer thickness.

The extent of the uptake of the active agent or combination of active agents from the quick dissolving oral mucosal drug delivery devices of the present invention at the site of application can be controlled by the dissolution/disintegration rate of the delivery device when applied to an oral mucosal surface. The delivery devices of the present invention release the active agent or combination of active agents contained therein as the delivery device dissolves or disintegrates over the release period. Once released from the delivery device, the active agent or combination of active agents may be absorbed by the mucosal tissue at or in proximity to the site of application or may be carried away to another location in the subject where it can be absorbed. For example, the active agent or active agents may be released by the delivery device into the mouth of a subject after which much of the active agent or combination of active agents is/are subsequently swallowed and taken up in the gastrointestinal tract. In contrast, the active agent or combination of active agents may be released by the delivery device into the mouth of a subject where the active agent or combination of active agents are largely absorbed through the surrounding oral mucosal tissue.

A further parameter affecting the uptake of an active agent or combination of active agents from the quick dissolving oral mucosal drug delivery devices of the present invention is the manner in which the active agent or combination of active agents is dispersed in the delivery device. For example, the active agent or combination of active agents may be dispersed as colloidal particles or be microencapsulated within the delivery device or alternatively may be mixed throughout the delivery device as a reagent during casting. In another example, the active agent can form a solid dispersion with a water soluble inert filler for purposes of increasing the solubility of the active agent when released from the inner layer thereby enhancing bioavailability of the active agent. This is exemplified here by sildenafil which is incorporated in a film with a water soluble inert filler, for example, xylitol, which has been found here to enhance the bioavailability of this agent. In another example, a bitter active agent can form saliva insoluble complex with water insoluble polymers for the purpose of taste masking.

One skilled in the art given the above description of the quick dissolving oral mucosal drug delivery devices of the present invention will be able to produce those devices using a variety of known processing methods. Preferably, the delivery devices of the present invention may be produced as follows. For example, the mucosal surface-coat-forming inner layer may be produced using solvent casting methods (including spray, draw, cast and curtain coating processes) and/or extrusion methods (including cold, warm and hot melt extrusion processes). The moisture barrier coating layers may be applied to the mucosal surface-coat-forming inner layer using roller coating, spraying, dipping and laminating with pre-formed outer layers (see FIGS. 4 to 7).

### EXAMPLES

### Examples 1-3

### Mucosal Surface-Coat-Forming Inner Layers, Compositions and Associated Properties

The mucosal surface-coat-forming inner layers were prepared as follows: a homogeneous mixture of ingredients was prepared in a coating solution in the amounts indicated in Table 1. The amounts are given as percentage on a weight of coating solution basis. The mixture was degassed in a vacuum chamber and coated on the non-siliconized side of a polyester film and dried in a hot air circulating oven to form a self supporting non-tacky and flexible inner layer. The inner layers were then cut into unit portions.

TABLE 1
Formulation of quick dissolving films
using several different hydrocolloids

| Composition: coating solution % | Ex. 1 | Ex. 2 | Ex. 3 |
|---|---|---|---|
| Pullalan (P-20) w % | | 17.5 | |
| Methocel E5 w % | 21.06 | | |
| POLYOX WSR N-10 w % | | | 1.8 |
| PVA (Vinol 125) w % | | 1.5 | |
| Cellulose gum w % | | | 8.0 |
| Propylene glycol w % | 1.0 | | 2.5 |
| Aspartame w % | 0.8 | 0.475 | 0.46 |
| Peppermint w % | 1.0 | 1.0 | 0.6 |
| Citric acid w % | 0.7 | 0.8 | |
| Cremphor EL4O w % | 1.0 | 1.0 | |
| Benzoic acid w % | 0.013 | 0.1 | 0.01 |
| FD&C blue #1 w % | qs. | | |
| FD&C yellow #5 w % | qs. | | |
| Ethanol w % | | 10.6 | |
| Water w % | 74.42 | 67.025 | 85.6 |

TABLE 2
Properties of the film formed from the coating solution of Table 1

| Properties of dry film | Ex. 1 | Ex. 2 | Ex. 3 |
|---|---|---|---|
| Thickness (mil) | 2.1 | 2.5 | 2.6 |
| Water content % | 1.7 | 8.5 | 8.0 |
| Dry tack (g) | 0.67 | 0.55 | 0.60 |
| Wet tack (g) | 60.16 | 86.64 | 72.27 |
| Tensile strength (psi) | 5242 | 2381 | 2036 |
| % Elongation (sec) | 2.9 | 4 | 2.9 |
| Modulus (psi) | 266834 | 272502 | 44566 |
| Tear resistance (N) | 0.02 | 0.16 | 0.01 |
| Disintegration (sec) | 12 | 20 | 12 |
| Dissolving time (sec) | 41 | 60 | 39 |

TABLE 3
Dry weight % for components of
Example I according to Tables 1 and 2

| Ingredients | Percentage (w/w) |
|---|---|
| Methocel E5 | 82.35 |
| Propylene glycol | 3.91 |
| Aspartame | 3.13 |
| Citric acid | 2.74 |
| Peppermint oil | 3.91 |
| PEG-40 Hydrogenated castor oil | 3.91 |
| Benzoic acid | 0.5 |
| FD&C blue #1 | qs. |
| FD&C yellow #5 | qs. |

TABLE 4
Mean values for parameters according to Example 1 in Table 1

| Properties | Value | ±SD (n) | |
|---|---|---|---|
| Weight (g/dosage film) | 0.028 | 0.001 | (4) |
| Thickness (mil) | 2.1 | 0.12 | (3) |
| pH | 3.07 | | (1) |
| Density (g/cm³) | 1.0485 | 0.009 | (3) |
| % Water content | 1.7 | 0.24 | (2) |
| Dry tack (g) | 0.674 | 0.110 | (6) |
| Wet tack (g) | 60.169 | 11.680 | (6) |
| Tensile strength (psi) | 5242 | 379 | (5) |
| % Elongation | 2.9 | 0.4 | (5) |
| Modulus (psi) | 266834 | 7910 | (5) |
| Tear-propagation resistance (N) | 0.02 | 0.00 | (4) |
| Disintegration time (sec) | 12 | 1 | (3) |
| Dissolving time (sec) | 41 | 5 | (3) |

### Examples 4-8

### Mucosal Surface-Coat-Forming Inner Layers Containing Hydroxypropylmethylcellulose and Therapeutic Agents.

The mucosal surface-coat-forming inner layers were prepared according to Examples 1-3. Therapeutic agents were added to the homogeneous mixture (coating solution) prior to forming the mucosal surface-coat-forming inner layer. FIG. 8 graphically illustrates the release profile of the four active agents incorporated into the mucosal surface-coat-forming inner layers according to Examples 5-8.

TABLE 5

| Composition | | | | | |
|---|---|---|---|---|---|
| (coating solution) | Ex. 4 | Ex. 5 | Ex. 6 | Ex. 7 | Ex. 8 |
| Nicotine | | 1.4 | | | |
| Hydromorphone | | | 2.92 | | |
| Oxybutynin | | | | 3.71 | |
| Estradiol | | | | | 1.49 |
| Peppermint | 1.0 | 1.0 | 1.0 | 1.0 | 1.0 |
| Methocel E5 (HPMC) | 21.06 | 21.06 | 21.06 | 21.06 | 21.06 |
| Propylene glycol | 1.0 | 1.0 | 1.01 | 1.0 | 1.0 |
| Aspartame | 0.8 | 0.8 | 0.8 | 0.8 | 0.8 |
| Citric acid | 0.7 | 0.7 | 0.7 | 0.7 | 0.7 |
| Cremphor EL40 | 1.0 | 1.0 | 1.0 | 1.0 | 1.0 |
| Benzoic acid | 0.013 | 0.013 | 0.013 | 0.013 | 0.013 |
| FD&C blue #1 | qs. | | | | |
| FD&C yellow #5 | qs. | | | | |
| Water | 74.43 | 73.03 | 71.51 | 70.72 | 72.94 |

TABLE 6
Properties of the film formed according
to the formulation in Table 5

| Properties | Ex. 4 | Ex. 5 | Ex. 6 | Ex. 7 | Ex. 8 |
|---|---|---|---|---|---|
| Thickness | 3.0 | 2.9 | 2.9 | 3.2 | 2.7 |
| (mil) | | | | | |
| Density | 1.18 | 1.19 | 1.13 | 1.20 | 1.16 |
| (g/cm³) | | | | | |
| Water | 1.8 | 2.93 | 2.42 | 2.32 | 2.31 |
| content % | | | | | |
| Dry tack (g) | 0.67 | 0.608 | 0.619 | 1.215 | 0.671 |
| Wet tack (g) | 49.08 | 54.81 | 84.34 | 88.85 | 39.91 |
| Tensile | 4393 | 3373 | 4138 | 3549 | 3688 |
| strength (psi) | | | | | |
| % | 8.3 | 8.3 | 7.6 | 8.1 | 7.5 |
| Elongation | | | | | |
| (sec) | | | | | |
| Modulus | 45969 | 48168 | 42110 | 41745 | 53334 |
| (psi) | | | | | |
| Tear | 0.03 | 0.02 | 0.01 | 0.03 | 0.01 |
| resistance | | | | | |
| (N) | | | | | |
| Disintegra- | 43.0 | 34.3 | 27.3 | 36.0 | 55.7 |
| tion (sec) | | | | | |
| Dissolving | 73.7 | 64.3 | 58.0 | 65.7 | 111.3 |
| time (sec) | | | | | |

TABLE 7
Composition of the Sildenafil film (% wet base)

| Composition | Percentage |
|---|---|
| Sildenafil citrate | 28.93 |
| Xylitol | 3.21 |
| Methocel E 15 | 4.59 |
| Propylene Glycol | 3.67 |
| Aspartame | 0.46 |
| Benzoic acid | 0.0045 |
| peppermint oil | 0.46 |
| Sodium EDTA | 0.0045 |
| Polyoxamer L-44 | 2.3 |
| Water | 55 |
| polypro 5000 | 0.92 |

TABLE 8
Properties of the film formed according
to the formulation in Table 7

| Properties | Ex.9 |
|---|---|
| Thickness | 3.2 ± 0.1 |
| Density (g/cm³) | 1.230 |
| Dry tack (g) | 1.21 ± 0.19 |
| Wet tack (g) | 23.79 ± 3.45 |
| Tensile strength (psi) | 42149 |
| % Elongation | 4.0 ± 0.7 |
| Modulus (psi) | 31822 ± 6137 |
| Tear resistence (N) | 0.04 ± 00 |
| Disintegration (sec) | 8.3 ± 1.5 |
| Dissolution (sec) | 23.7 ± 1.5 |

### Example 9

### A Comparison of Properties of Mucosal Surface-Coat-Forming Inner Layers Using Different Hydroxypropylmethylcellulose Polymers.

The properties of a mucosal surface-coat-forming inner layer according to the present invention may be modified by varying the individual components used therein. For example, the dissolution rate of the film may be prolonged by using hydroxypropylmethylcellulose (HPMC) with higher molecular weight as shown below in Table 9.

TABLE 9a
Properties of selected commercial
hydroxypropylmethylcellulose polymers

| | Methocel Type (Dow Pharmaceuticals) | | | | | | |
|---|---|---|---|---|---|---|---|
| Property | E3 | E5 | K3 | E15 | A15 | E50 | F50 |
| % Methoxyl | 29 | 29 | 22 | 29 | 30 | 29 | 28 |
| % Hydroxy- | 8.5 | 8.5 | 8.1 | 8.5 | 0 | 8.5 | 5.0 |
| propyl | | | | | | | |
| Viscosity | 2-4 | 4-6 | 2-4 | 12-18 | 12-18 | 40-60 | 40-60 |
| 2% (cps) | | | | | | | |

* Each value is the mean S ± D, n = 6

TABLE 9b
Properties of films prepared according to Example 1, using different hydroxypropylmethylcellulose polymers

| Property | E3 | E5 | K3 | E15 | A15 | E50 | F50 |
|---|---|---|---|---|---|---|---|
| Dry tack (g) | 0.61 ± 0.08 | 0.67 ± 0.110 | 0.82 ± 0.12 | 0.66 ± 0.09 | 0.52 ± 0.09 | 0.68 ± 0.14 | 0.52 ± 0.12 |
| Wet tack (g) | 93.4 ± 8.95 | 60.169 ± 11.6 | 60.2 ± 8.77 | 65.4 ± 17.8 | 18.4 ± 3.0 | 79.1 ± 17.1 | 64.1 ± 11.2 |
| Tensile strength | 1921 ± 442 | 5242 ± 379 | 2043 ± 268 | 4316 ± 384 | 3351 ± 165 | 3725 ± 123 | 3905 ± 590 |
| (psi) | | | | | | | |
| % Elongation | 4.2 ± 1.2 | 2.9 ± 0.4 | 3.8 ± 0.8 | 16.9 ± 4.3 | 11.1 ± 2.4 | 11.4 ± 2.4 | 15.0 ± 3.4 |
| Modulus (psi) | 44368 ± 864 | 266834 ± 79 | 41737 ± 816 | 46889 ± 416 | 35914 ± 964 | 41651 ± 282 | 43644 ± 942 |
| Tear resistence | 0.040.01 ± | 0.02 ± 0 | 0.05 ± 0.01 | 0.09 ± 0.03 | 0.12 ± 0.02 | 0.05 ± 0.01 | 0.08 ± 0.01 |
| (N) | | | | | | | |
| Disintegration | 17.0 ± 4.4 | 12 ± 1 | 15.3 ± 1.5 | 21.9 ± 1.6 | 161.0 ± 15.9 | 33.2 ± 5.1 | 24.1 ± 1.3 |
| (sec) | | | | | | | |
| Dissolution | 35.7 ± 2.1 | 41 ± 5 | 31.0 ± 1.0 | 51.6 ± 1.3 | >600 | 71.6 ± 3.3 | 62.1 ± 2.8 |
| (sec) | | | | | | | |

### Examples 10-15

### Moisture Barrier Coating Layers, Compositions and Associated Properties

Preferred moisture barrier coating layers were prepared according and applied to a mucosal surface-coat-forming inner layer of the present invention according to Examples 11-15. Table 10 lists the wettability of the moisture barrier coating layers produced in Examples 11-15. Specifically, the wettability of these moisture barrier-coating layers was determined using wettability markers (30 to 42 dyne/cm). Table 11 compares the physical properties of a mucosal surface-coat-forming inner layer with and without a moisture barrier coating as described in Example 15. FIG. 9 graphically illustrates the moisture uptake of the moisture barrier coating layers of Examples 10-15.

(A) Example 10 (mucosal surface-coat-forming inner layer without a moisture barrier):

(1) wetted 105.0 grams of hydroxypropylmethylcellulose (Methocel E5 commercially available from Dow Pharmaceuticals) with 50.0 grams of ethanol;

(2) dissolved 5.0 grams of propylene glycol, 5.0 grams of peppermint, 5.0 grams polyoxyl 40 hydrogenated castor oil (i.e., Cremaphore RH40), 2.4 grams Aspartame 2.5 grams of acesulfame potassium, 2.5 grams of citric acid, 0.005 grams of sodium EDTA, 0.1 grams of methylparaben, 0.034 grams of FD&C Blue 1 and Yellow 5 in 322.32 grams of water;

(3) homogenized the product of (2);

(4) added the product of (1) into the product of (3) with vigorous adgitation until the Methocel E5 completely dissolved into aqueous solution;

(5) degassed the product of (4);

(6) casted the product of (5) at 10 mil thickness onto a casting liner; and,

(7) dried the product of (6) in an oven, removing the solvent to form a mucosal surface-coating-forming inner layer.

(B) Example 11:

(1) dissolved 3.00 grams of polyethylene glycol 8000 and 2.00 grams of white beewax in 45 ml of warm ethanol;

(2) sprayed the product of (1) on one side of the mucosal surface-coat-forming inner layer of the present invention (i.e., the mucosal surface-coating-forming inner layer of Example 10); and,

(3) dried the product of (2) in an oven to remove the ethanol.

(C) Example 12:

(1) dissolved 3.00 grams of polyethylene glycol 8000 and 2.00 grams of emulsifying wax in 45 ml of warm ethanol;

(2) sprayed the product of (1) on one side of the mucosal surface-coat-forming inner layer of the present invention (i.e., the mucosal surface-coating-forming inner layer of Example 10); and,

(3) dried the product of claim (2) in an oven to remove the ethanol.

(D) Example 13:

(1) dissolved 3.00 grams of polyethylene glycol 8000 and 2.00 grams of Pluornic F68 in 45 ml of warm ethanol;

(2) sprayed the product of (1) on one side of the mucosal surface-coat-forming inner layer of the present invention (i.e., the mucosal surface-coating-forming inner layer of Example 10); and,

(3) dried the product of claim (2) in an oven to remove the ethanol.

(E) Example 14:

(1) dissolved 1.00 grams of polyethylene glycol 8000 and 4.00 grams of Pluronic F87 in 45 ml of warm ethanol;

(2) sprayed the product of (1) on one side of the mucosal surface-coat-forming inner layer of the present invention (i.e., the mucosal surface-coating-forming inner layer of Example 10); and,

(3) dried the product of claim (2) in an oven to remove the ethanol.

(F) Example 15:

(1) dissolved 5.00 grams of polyethylene glycol 8000, 20.00 grams of Pluronic F87, 1.25 grams of butylated hydroxyanisole and 1.50 grams of peppermint oil in 230 grams of warm ethanol;

(2) sprayed the product of (1) on one side of the mucosal surface-coat-forming inner layer of the present invention (i.e., the mucosal surface-coating-forming inner layer of Example 10); and,

(3) dried the product of claim (2) in an oven to remove the ethanol.

TABLE 10
wettability data comparison

| Moisture barrier layer composition | Wettability (dyne/cm) |
|---|---|
| Example 10 (without coating) | 36-38 |
| Example 11 | 30-32 |
| Example 12 | 32-34 |
| Example 13 | 36-38 |
| Example 14 | 34-36 |

TABLE 11
Comparison of physical properties
with and without barrier layer coating

| | Without moisture barrier | With moisture barrier |
|---|---|---|
| Property | layer (Example 10) | layer (Example 15) |
| Thickness (mil) | 1.06 ± 0.09 | 1.29 ± 0.08 |
| Weight (mg) | 17.61 ± 0.66 | 19.83 ± 0.48 |
| Tensile strength | 4928.82 ± 714.70 | 6582.40 ± 926.93 |
| (psi) | | |
| % elongation | 4.78 ± 1.22 | 5.20 ± 0.86 |
| Modulus (psi) | 261117.20 ± 21544.26 | 336147.54 ± 9270.31 |
| Disintegration time | 4.3 ± 1.03 | 5 ± 1.09 |
| (sec) | | |

### Examples 16-23

Provide various examples of Mucosal surface-coat-forming inner layers using different ingredients. The mucosal surface-coat-forming inner layers of Examples 16-23 were prepared in the same fashion as those described in Examples 1-3. The specific ingredients and amounts used for each of Examples 16-23 are listed in Table 12.

TABLE 12
Mucosal surface-coat-forming inner layer compositions

| | Ex. | Ex. | Ex. | Ex. | Ex. | Ex. | Ex. | Ex. |
|---|---|---|---|---|---|---|---|---|
| Composition in coating solution | 16 | 17 | 18 | 19 | 20 | 21 | 22 | 23 |
| Famotidine | 5.23 | | | | | | | |
| Granisetron | | 3 | | | | | | |
| Hydrocortisone | | | 0.47 | | | | | |
| Loratadine | | | | 7.66 | | | | |
| Vinpocetine | | | | | 2.43 | | | |
| Buprenorphine | | | | | | 1.7 | | |
| Domperidone | | | | | | | 3.75 | |
| Loperamide | | | | | | | | 3 |
| Methocel E15 | 2.95 | | | | | | | |
| Methocel E5 | | | 43.2 | | | | 16.3 | 13 |
| Methocel E3 | | 15 | | 15.5 | | | | |
| Methocel E50 | | | | | 10.6 | 6.17 | | |
| Eudragit L100 | 10.5 | | | | | | | |
| Strawberry twist | 0.35 | 0.5 | 1.33 | | | | 0.5 | 0.5 |
| Peppermint oil | 0.3 | | | 2.81 | | 0.5 | | |
| Cherry black | | | | | 0.34 | | | |
| Bitterness suppressor | | | | | 1.29 | | | |
| Cremophore RH40 | 0.08 | 0.25 | 0.66 | 0.94 | 0.29 | 0.17 | 0.25 | 0.25 |
| Ethanol | 4.93 | 25 | 13.3 | 31.8 | 12.1 | 40.1 | 25 | 25 |
| Propylene glycol | | 2.0 | 19.9 | 3.47 | 4.13 | 0.5 | 2.5 | 2.0 |
| 70% sorbitol | 15 | | | | | | | |
| Aspartame | 0.27 | 0.75 | 2.00 | | | | 0.75 | 0.75 |
| Acesulfame K | 0.28 | 0.75 | 2.00 | | 1.82 | 1.08 | 0.75 | 0.75 |
| Neohesperidine | 0.1 | | | 0.1 | | 0.1 | | |
| Glycyrrhizin | | | | 5.58 | 12.2 | | | |
| Sodium EDTA | 0.04 | 0.13 | 0.33 | 0.03 | 0.05 | 0.01 | 0.13 | 0.13 |
| Talc | | | | 0.2 | | | | |
| Sodium carbonate | 0.16 | | | | | 0.2 | | |
| Maltose | | | | | | 4.74 | | |
| Methylparaben/Propylparaben | 0.03 | 0.06 | 0.17 | 0.02 | 0.02 | 0.01 | 0.06 | 0.06 |
| FD&C Red #40 | 0.01 | 0.01 | 0.01 | | 0.01 | | 0.01 | 0.01 |
| FD&C Yellow #5 | | | | 0.01 | | 0.01 | | |
| FD&C Blue #1 | | | | 0.01 | | 0.01 | | |
| Water | 59.8 | 52.5 | 16.7 | 31.8 | 54.7 | 44.7 | 50.0 | 54.5 |

The present invention having been disclosed in connection with the foregoing embodiments, additional embodiments will now be apparent to persons skilled in the art. The present invention is not intended to be limited to the embodiments specifically mentioned, and accordingly reference should be made to the appended claims rather than the foregoing discussion, to assess the spirit and scope of the present invention in which exclusive rights are claimed.

We claim:

1. A quick dissolving oral mucosal drug delivery device, comprising a mucosal surface-coat-forming inner layer disposed between two moisture barrier coating layers; wherein the mucosal surface-coat-forming inner layer comprises a water-soluble hydrocolloid and an active agent; and wherein the two moisture barrier coating layers comprise a non-crosslinked polymer and a moisture barrier modifier.

2. The drug delivery device of claim 1, wherein the mucosal surface-coat-forming inner layer further comprises a plasticizer.

3. The drug delivery device of claim 1, wherein the mucosal surface-coat-forming inner layer further comprises a detackifier.

4. The drug delivery device of claim 1, wherein the mucosal surface-coat-forming inner layer further comprises at least one member selected from the group consisting of taste modifying agents, emulsifying agents, buffering agents, coloring agents and preservatives.

5. The drug delivery device of claim 1, wherein the moisture barrier coating layers further comprise at least one member selected from the group consisting of flavoring agents and anti-oxidants.

6. The drug delivery device of claim 1, wherein the mucosal surface-coat-forming inner layer has a thickness i n the range of 1-50 mil.

7. The drug delivery device of claim 1, wherein the moisture barrier coating layers have a thickness in the range of 1 to 25 μm each.

8. The drug delivery device of claim 1, wherein the device dissolves or disintegrates in the oral cavity within 1-600 seconds.

9. The drug delivery device of claim 1, wherein the active agent is selected from the group consisting of a therapeutic agent, a dietary supplement and a hygiene aid.

10. The drug delivery device of claim 1, wherein the active agent is sildenafil citrate.

11. The drug delivery device of claim 1, wherein the active agent is selected from the group consisting of nicotine, hydromorphone, oxybutynine and estradiol.

12. The drug delivery device of claim 1, wherein the active agent is selected from the group consisting of famotidine, granisetron, hydrocortisone, loratadine, vinpocetine, buprenorphine, domperidone and loperamide.

13. A quick dissolving oral mucosal drug delivery device, comprising a mucosal surface-coat-forming inner layer disposed between two moisture barrier coating layers; wherein the mucosal surface-coat-forming inner layer comprises a water-soluble hydrocolloid, at least one active agent, at least one plasticizer and at least one detackifier; and wherein the two moisture barrier coating layers comprise at least one water soluble or dispersible film former and at least one moisture barrier modifier.

14. The drug delivery device of claim 13, wherein the mucosal surface-coat-forming inner layer further comprises at least one member selected from the group consisting of taste modifying agents, emulsifying agents, buffering agents, coloring agents and preservatives.

15. The drug delivery device of claim 13, wherein the moisture barrier coating layers further comprise at least one member selected from the group consisting of flavoring agents and anti-oxidants.

16. A method of making a quick dissolving oral mucosal drug delivery device, comprising:
(a) dissolving a hydrocolloid in a solvent to form a substantially homogeneous preparation;
(b) adding to (a), an active agent and at least one reagent selected from the group consisting of an emulsifier, a plasticizer, a taste modifier, a water soluble inert filler, a coloring agent, a preservative, a permeation enhancer, a stabilizer and a buffering agent to form a coatable or extrudable mixture;
(c) forming a mucosal surface-coat-forming inner layer from the mixture of (b); and,
(d) applying moisture barrier coating layers to the mucosal surface-coat-forming inner layer of (c).

17. A method for administering an active agent to a subject comprising:
(a) applying a quick dissolving oral mucosal drug delivery device to an oral mucosal surface of the subject;
wherein the quick dissolving oral mucosal drug delivery device comprises a mucosal surface-coat-forming inner layer disposed between two moisture barrier coating layers; wherein the mucosal surface-coat-forming inner layer comprises a water-soluble hydrocolloid, at least one active agent, at least one plasticizer and at least one detackifier; and wherein the two moisture barrier coating layers comprise at least one water soluble or dispersible film former and at least one moisture barrier modifier.

18. A quick dissolving oral mucosal drug delivery device, comprising a mucosal surface-coat-forming inner layer encapsulated within a moisture barrier coating layer; wherein the mucosal surface-coat-forming inner layer comprises a water-soluble hydrocolloid and an active agent; and wherein the moisture barrier coating layer comprises a non-crosslinked polymer and a moisture barrier modifier.

19. The drug delivery device of claim 18, wherein the moisture barrier coating layers further comprise at least one member selected from the group consisting of flavoring agents and anti-oxidants.

20. The drug delivery device of claim 18, wherein the moisture barrier coating layers have a thickness in the range of 1 to 25 μm each.

21. The drug delivery device of claim 18, wherein the device dissolves or disintegrates in the oral cavity within 1-600 seconds.

22. A quick dissolving oral mucosal drug delivery device, comprising a mucosal surface-coat-forming inner layer encapsulated by a moisture barrier coating layer; wherein the mucosal surface-coat-forming inner layer comprises a water-soluble hydrocolloid, at least one active agent, at least one plasticizer and at least one detackifier; and wherein the moisture barrier coating layer comprises at least one water soluble or dispersible film former and at least one moisture barrier modifier.

23. The drug delivery device of claim 22, wherein the moisture barrier coating layer further comprises at least one member selected from the group consisting of flavoring agents and anti-oxidants.
